(19) United States
(12) Patent Application Publication
(10) Pub. No.: US 2013/0095560 A1
(43) Pub. Date: Apr. 18, 2013

(54) Method of Producing Tissue Culture Media Derived from Plant Seed Material and Casting of Mycological Biomaterials

(71) Applicants: Gavin McIntyre, Troy, NY (US); Eben Bayer, Troy, NY (US); Christopher Scully, Troy, NY (US); Amanda Palazzolo, Ballston Spa, NY (US)

(72) Inventors: Gavin McIntyre, Troy, NY (US); Eben Bayer, Troy, NY (US); Christopher Scully, Troy, NY (US); Amanda Palazzolo, Ballston Spa, NY (US)

(21) Appl. No.: 13/618,261

(22) Filed: Sep. 14, 2012

Related U.S. Application Data
(60) Provisional application No. 61/573,888, filed on Sep. 14, 2011.

(51) Int. Cl. C12N 1/14

(52) U.S. Cl. CPC C12N 1/14
USPC 435/256.8

(57) ABSTRACT

A culture/gelling liquid is formed of a mixture of seed matter and water that is inoculated with a desired cell or tissue strain. The culture/gelling liquid is combined with a substrate in order to be formed into molds, and incubated until a member is formed. The substrate may also be pre-colonized.

This application claims the benefit of Provisional Patent Application 61/573,888 filed Sep. 14, 2011.

This invention relates to methods of producing tissue culture media derived from plant seed material. More particularly, this invention relates to methods of producing tissue culture media derived from plant seed material and leveraging the media to inoculate and cast an engineered substrate into a complex geometry. Still more particularly, this invention relates to the casting of mycological biomaterials.

As is known from published United States Patent Application 2008/0145577, use can be made of a fungus to form composite materials by mixing an inoculum including a preselected fungus with discrete particles and a nutrient material capable of being digested by the fungus.

It is an object of the invention to provide an economical method of producing a tissue culture media for use in making composite materials.

It is another object of the invention to provide an economical method for casting items from a tissue culture media.

It is another object of the invention to provide an economical method for casting engineered substrates from a tissue culture media.

Briefly the invention provides a method of making a tissue culture media comprising the steps of measuring a selected seed matter at specified ratio (m:v) with water to form a mixture; autoclaving the mixture in an autoclavable vessel; inoculating the autoclaved mixture with a cell or tissue strain; and incubating the inoculated mixture for a desired time to form a culture/gelling liquid.

The methods described within explain the practices and materials that may be used to produce tissue culture media comprised of plant seed material and water, as well as potentially supplemental nutrients (minerals, vitamins, and the like). Certain plant seeds and seed matter, upon mixing with water, gelatinize and the residual solids become suspended in solution. Such materials include chia seeds, basil seeds, psyllium seed husks, and the like. The seeds provide the essential nutritive and thickening properties for the media while minimizing the number of ingredients added to water. Industry has found use in purifying the gel-forming fraction of these substances for dietary supplementation. This tissue culture media may be used for applications that currently employ media requiring multiple costly ingredients and chemical inputs.

### BACKGROUND

Scientific literature has reported the successful use of fatty acids in promoting growth of mycelial tissue liquid cultures. The purported mechanism of action for this achievement may be attributed to the uptake of lipids to the cell membranes that then facilitate nutrient transfer from media into cells. Previously work has proven this effective in liquid cultures of yeast, bacteria, and fungal mycelia.

This is relevant to the use of seeds in tissue culture media because seeds are often a valuable source of oils and fatty acids in addition to nutrients and other beneficial compounds (carbohydrates, minerals, and simple sugars). For example, chia seeds have been reported to contain 25-38% oil by weight. These are mostly polyunsaturated fatty acids (alpha-linoleic acid making up 62% of total fatty acids).

Chia seeds, like many other seeds, possess additional nutritive properties that are quite valuable for cell growth and proliferation. The seeds are 19-23% protein and 22.1% dietary fiber. In vivo digestibility has been rated at 93-100%. Several types of antioxidants are present, such as myricetin, quercetin, kaempferol, caffeie acid, and chlorogenic acid. They also provide B vitamins, calcium, phosphorus, potassium, zinc, magnesium, and copper.

### Applications

1. Tissue Culture Media
a. Current conventional methods for producing cell or tissue culture media entail the composition of multiple chemical-based ingredients. These include gelling agents, nutrients, and antimicrobials. Most of these ingredients require considerable preprocessing and can become prohibitively expensive at scale.
b. There are many select materials from plants, algae and animals that may be used in substitution of all the above-mentioned ingredients. Certain plant seeds, hulls, husks, and other matter are all possibilities. Alginates derived from algae can be used to enhance the viscosity of the culture media, either solely or in conjunction with the seed matter. Similarly, gelatin can serve in a manner as the alginates. These materials provide nutrients and gelling properties and require virtually no pre-processing. They may simply be added to water at a specific concentration to become functional as tissue culture media. The nutritive elements are essential for producing viable, healthy tissue. The gelling properties are vital for cell adhesion and growth. The enhanced viscosity of these select substances is tolerant of heat sterilization, an essential step for creating pure cultures.
c. The current basis for previously developed culture media utilizes alginates and agars as a basis for enhanced viscosity. Though this is effective and stable through sterilization, there is no solid on which the culture may adhere to and proliferate. A unique aspect of media constructed from seeds is the presence of scaffolding on which the tissue may adhere to while growing. In this way, the seeds also act as a carrier for subsequent inoculation.
d. There are many possibilities for grinding seeds and hulls to attain greater surface area and also the release of nutrients from whole seed. Grinding is a step that may be done in-house, and is easily customizable for specific applications. For example, finely ground chia seeds may be used as a base for the media while coarsely ground chia seeds may be used as a carrier using various combinational ratios thereof. A lower mass of ground chia seeds has also been found to achieve a similar suspension viscosity as whole seed; 40 g of ground seed in comparison to 60 g of whole seed with each suspend an entire liter of water.

2. Gel Assisted Casting of Mycological Substrate
a. Casting of mycological substrate is a process used to form substrate (scaffolding) on which fungal mycelium may grow to produce a bonded shape. Casting is often aided by the use of a gelling agent. The above-mentioned media concept provides many valuable properties to be ideal for this application. In some cases, the gelling agent provides both a means of substrate inoculation (the point from which mycelial culture will colonize and bond the substrate) as well as enhanced substrate binding. The gelling agent may also be used simply as a binding agent, supplemental to an inoculum (grain spawn, liquid tissue suspension, and the like). The benefits to be gained from the seed media in this application include provision of nutrients to improve growth characteristics and gelling of substrate to improve feature resolution and retention prior to complete colonization. The seeds may act as “carriers” to evenly distribute the culture within the substrate as uniform inoculation points.

### Process Steps

### Tissue Culture

1. Measure selected seed matter, alginate, gelatin or a combination thereof at a specified ratio (m:v) with water.
2. Add pre-measured seed matter and water to autoclavable vessel.
3. Autoclave the resulting substance accordingly.
4. Inoculate with desired cell or tissue strain.
5. Allow to incubate for desired time frame.

### Mycological Substrate Casting Aid

1. Measure selected seed matter, alginate, gelatin or a combination thereof at specified ratio (m:v) with water.
2. Add pre-measured seed matter and water to autoclavable vessel.
3. Autoclave the resulting substance accordingly.
   Option A: Inoculate with desired cell or tissue strain. Allow to incubate for desired time frame. Inoculate substrate.
   Option B: Inoculate substrate as desired. Add seed matter media.
4. Cast combined materials into desired form. Incubate for desired time frame.

### EXAMPLE 1

### Liquid-Inoculated Substrate

In this example, liquid addition to substrate serves, two primary functions: introduction of mycelial culture to the substrate and introduction of gelling agent to the substrate.
I. The media is composed of one or more components from one or more of the three columns in Table x below with water in an Erlenmeyer flask. For example, the media may be composed of flax seeds, poppy seeds, manganese, and psyllium husks; just chia seeds; or yeast with guar gum. This includes various ratios and concentrations of each, and should be adjusted for desired moisture content, growth characteristics, and mycelium species. The flask opening is covered with aluminum foil that is compressed around the edges of the opening.

TABLE X
Liquid Inoculum Components for Gel Assisted Casting

| A) Seeds | B) Supplemental Nutrition | C) Gelling Agent |
|---|---|---|
| Chia | Yeast | Psyllium Husks |
| Flax | Calcium | Gelling seeds (such as chia) |
| Sesame | Magnesium | Agar |
| Mustard | Complex Carbohydrates | Protein (such as guar) |
| Poppy | Manganese | Starches (such as corn |
| | | starch) |

2. The flask is sterilized in a pressure cooker at 15 psi and 240° F. for 60 minutes.
3. Once cooled to room temperature, the flask is sprayed with 70% isopropanol and placed in a laminar flow hood with a sterile Eberbach blender. One 10cm-diameter Petri dish culture of Ganoderma applanatum (or equivalent) mycelium is aseptically added to the blender with the media and homogenized. The inoculated media is added back to the flask and agitated to distribute. This is the culture/gelling liquid.
4. The flask is then incubated at ambient conditions for seven days.
5. The substrate is prepared by combining 5 L oat hulls (or equivalent), water, maltodextrin, and CaSO₄ in an autoclavable plastic bag. Water, maltodextrin, and CaSO₄ concentrations and ratios are adjusted for desired moisture content, growth characteristics and mycelium species. The substrate is sterilized at 15 psi and 240° F. for 60 minutes in a pressure cooker.
6. Once cooled, the substrate is sprayed with 70% isopropanol and placed in a laminar flow hood with the culture/gelling liquid, molds, and sterile cell culture incubator which controls relative humidity, carbon dioxide and temperature. The culture/gelling liquid is added to the substrate at a rate of 20% volume:volume (1 L culture:5 L substrate). After mixing, the substrate is formed in molds, released, and then incubated in a cell culture incubator at 95% relative humidity, 0.5% carbon dioxide and 25° C. for 6 days.
7. The part is then removed from the cloner and dried to 0% moisture.

### EXAMPLE 2

### Grain-Inoculated Substrate

In this example, a liquid solution of nutritional supplements and gelling agents is added to a substrate with grain inoculum.
1. The nutritional gelling solution is produced by combining one or more components from one or more columns in Table x above with water in an Erlenmeyer flask. The flask opening is covered with aluminum foil that is [symbol]
2. covered around the edges of the opening.
3. The flask is sterilized in a pressure cooker at 15 psi and 240° F. for 60 minutes.
3. Once cooled to room temperature, the flask is sprayed with 70% isopropanol and placed in a laminar flow hood with sterile substrate (prepared as in Example I), Ganoderma applanatum (or equivalent) grain inoculum, molds, and sterile cell culture incubators. The inoculum is added to the substrate at a rate of 360 g per 5 L substrate. The nutritional gelling solution is added to the inoculated substrate at a rate of 20% volume:volume (IL solution:5 L substrate).
4. The substrate is formed in molds, released, and then incubated in a cell culture incubator at 95% relative humidity, 0.5% carbon dioxide and 25° C. for 6 days.
5. The part is then removed from the incubator and dried to 0% moisture.

### EXAMPLE 3

### Live, Pre-Colonized Substrate

In this example, a liquid solution of nutritional supplements and gelling agents is added to live, pre-colonized substrate.
1. The nutritional gelling solution is produced by combining one or more components from one or more columns in Table x above with water in an Erlenmeyer flask. The flask opening is covered with aluminum foil that is covered around the edges of the opening.
2. The flask is sterilized in a pressure cooker at 15 psi and 240° F. for 60 minutes.
3. Once cooled to room temperature, the flask is sprayed with 70% isopropanol and placed in a laminar flow hood with live, precolonized substrate (grain- or liquid-inoculated substrate prepared as in Example I or 2, respectively,—without gelling solution—and incubated for 5 days in bulk), and sterile hydroponic cell culture incubators. The pre-colonized substrate is ground and combined with the nutritional gelling solution at a rate of 20% volume:volume (IL solution:5 L substrate).
4. The substrate is formed in molds, released, and then incubated in a cell culture incubator at 95% relative humidity, 0.5% carbon dioxide and 25° C. for 6 days.
5. The part is then removed from the incubator and dried to 0% moisture.

### EXAMPLE 4

### Desiccated, Pre-Colonized Substrata

In this example, a liquid solution of nutritional supplements and gelling agents is added to live, pre-colonized substrate.
1. The nutritional gelling solution is produced by combining one or more components from one or more columns in Table x above with water in an Erlenmeyer flask. The flask opening is covered with aluminum foil that is covered around the edges of the opening.
2. The flask is sterilized in a pressure cooker at 15 psi and 240° F. for 60 minutes.
3. Once cooled to room temperature, the flask is sprayed with 70% isopropanol and placed in a laminar flow hood with desiccated, pre-colonized substrate (grain- or liquid-inoculated substrate as prepared in Example 1 or 2, respectively,—without gelling solution—and incubated in a tool for 6 days, then dried to 10-50% moisture and ground to particles), and sterile cell culture incubators The pre-colonized substrate is ground and combined with the nutritional gelling solution at a rate of 20% volume:volume (IL solution:51 substrate).
4. The substrate is formed in molds, released, and then incubated in a cell culture incubator at 95% relative humidity, 0.5% carbon dioxide and 25° C. for 6 days.
5. The part is then removed from the incubator and dried to 0% moisture.

A mixture formed of a culture/gelling liquid and substrate described above may be used in the fabrication of various members, for example, using various types of molding techniques. For example, one method that could be employed is the use of high pressure to compact the materials together. Much in the same way as structural wood composites, such as Medium Density Fiberboard (MDI′) and Oriented Strand Board (OSB), desired shapes could be created using compression to create cohesion between particles or fibers.

The use of high pressure, heat, gels, and waxes or adhesive additives could prove quite useful in primary shaping of a mycological composite product. This would produce a high density part, and due to its small or nonexistent gaps between particles, additional additives or created voids may be needed to allow for respiration.

Temperature changes could be employed to activate temperature set binding agents, such as waxes, however the nutritional agents in the biological substrate may be enough to hold certain shapes together until the fungi fully colonize the part. Steam could be used to activate binding agents as well as clean machined cavities that would serve as molds.

With a highly cohesive substrate, or one with long fibers, a small amount of pressure may be able to cause structural cross-linking of particles or fibers enough to hold a desired shape. Various additives have already proven to serve as functional binding agents including guar gum, heated starch solutions, and chia, seeds. This is referred to as gel-assisted casting. Hand-packing substrate coated in guar gum and calcium into a soft-walled tool and then ejecting the finished member by hand has shown the ability to create a self-supported 3D casted part that over about 5 days became fully colonized with mycelia.

One method of producing such a self-supporting part would be to start with a two part mold, such as a die used in the practice of die-casting of aluminum, or plastic injection molding. This mold could then be closed, and through an inlet cavity, filled with either a ground pre-colonized substrate or a freshly inoculated substrate by way of gravity, pneumatic or hydraulic filtration and mechanical conveyance, such as a peristaltic or piston pump.

In the case of hydraulic or pneumatic conveyance, the cavities could be perforated, and the die could be enclosed in an air-tight container. By removing fluid from the container and negatively pressurizing the container, fluid would rush out of the cavity, pulling in fluid through the injection port, which would in turn pull substrate into the cavity and against the walls of the cavity of the mold. The walls would filter out the substrate from the fluid, which could be either a gas or liquid, and compact the substrate together under the pressure of the flow.

After the cavity is filled by any of the methods described, additional pressure could be applied to the mixture of substrate within the cavities by way of a piston compressing additional material in through the injection port. After the cavity has been sufficiently filled and compacted, the part could be removed by separating the two part die and ejecting the part onto a sanitary conveyor by pushing pressurized air through the holes in the mold.

Use may also be made of a three part tool (not shown) comprised of three independently actuated featured cavities. Two parts form a cavity which is the negative of the desired geometry and the third part serves as a sleeve to affix the two negative-forming parts together. In this embodiment, sterilized and inoculated substrate is applied to the cavity and mechanically actuated to uniformly fill the void and/or modify the density. The material could be compressed with a piston or vibrated into position. After casting of the substrate into a coherent mass, the sleeve is removed from the tool and the two negative-forming parts are retracted to expose the casted substrate. The casted part is then conveyed into an incubation environment or incubation vessel that can control environmental conditions including but not limited to temperature, CO2, and relative humidity.

In another embodiment, use is made of a two part tool wherein the two parts form a cavity which is the negative of the desired geometry and the tool is conveyed on a work surface, such as a conveyor belt. Such a tool may employ injection molding techniques wherein a slurry of the mixture of culture/gelling liquid and inoculated substrate is pumped with a metering pump (peristaltic, diaphragm) into the cavity of the tool. Also, the casting cavity may have multiple chambers to form an engineered substrate of complex geometry. After casting of the substrate into a coherent mass, the sleeve is removed from the tool and the two negative-forming parts are retracted to expose the casted substrate. The casted part is then conveyed into an incubation environment.

Multiple parts may be cast simultaneously, conveyed forward and then pushed into incubation vessels.

Of note, depending of the constituents, a mixture of culture/gelling liquid and inoculated substrate placed in the cavity of a casting tool and held for a time sufficient for the mixture to form a coherent mass that may be subsequently processed out of the tool. For example, some mixtures need beheld for several seconds whereas other mixtures may take several hours. Once removed from the casting tool, the coherent mass is incubated and then dried.

The invention thus provides an economical method of producing a tissue culture media for use in making composite materials.

The invention provides improved methods for casting mycological biomaterials particularly from tissue culture media derived from plant seed material.

What is claimed is:

1. A method of making a tissue culture comprising the steps of measuring a selected seed matter at specified ratio (m:v) with water to form a mixture;
autoclaving the mixture in an autoclavable vessel;
inoculating the autoclaved mixture with one of a cell strain and tissue strain; and
incubating the inoculated mixture for desired time to form a culture/gelling liquid.

2. A method as set forth in claim 1 wherein said selected seed matter is selected from the group consisting of chia, flax, sesame, mustard and poppy.

3. A method as set forth in claim 1 wherein said cell strain and tissue strain is a culture of Ganoderma applanatum.

4. A method comprising the steps of
forming a tissue culture media composed of water, a gelling agent, at least one of seeds selected from the group consisting of chia seeds, flax, sesame, mustard and poppy and a supplemental nutrient selected from the group consisting of yeast, calcium, magnesium, complex carbohydrates and manganese;
sterilizing said media at a predetermined temperature and for a predetermined time;
cooling said sterilized media to room temperature;
inoculating said cooled media with a culture of Ganoderma applanatum to form a culture/gelling liquid;
forming a substrate composed of oat hulls, water, maltodextrin and CaSO₄;
sterilizing said substrate and cooling said sterilized substrate;
combining said culture/gelling liquid and said substrate to form a mixture;
placing said mixture in a mold of predetermined shape;
incubating said mixture to form a member of predetermined shape; and
drying said member to 0% moisture.

5. A method as set forth in claim 5 wherein said culture/gelling liquid is combined with said substrate at a ratio of 20% on a volumetric basis.

6. A method as set forth in claim 5 wherein said culture/gelling liquid is combined with said substrate at a ratio of 1 liter of culture/gelling liquid to 5 liters of substrate.

7. A method comprising the steps of
forming a tissue culture media composed of water, a gelling agent, at least one of seeds selected from the group consisting of chia seeds, flax, sesame, mustard and poppy and a supplemental nutrient selected from the group consisting of yeast, calcium, magnesium, complex carbohydrates and manganese;
sterilizing said media at a predetermined temperature and for a predetermined time;
cooling said sterilized media to room temperature;
forming a substrate composed of oat hulls, water, maltodextrin and CaSO₄;
combining said culture/gelling liquid and said substrate to form a mixture;
inoculating said mixture with a Ganoderma applanatum grain inoculum;
placing said inoculated mixture in a mold of predetermined shape;
incubating said mixture to form a member of predetermined shape; and
drying said member to 0% moisture.

8. A method as set forth in claim 7 wherein inoculum is added to said substrate at a ratio of 360 grams per 5 liters of substrate and said culture/gelling liquid is combined with said substrate at a ratio of liter of culture/gelling liquid to 5 liters of substrate.

9. A method comprising the steps of
forming a tissue culture media composed of water, a gelling agent, at least one of seeds selected from the group consisting of chia seeds, flax, sesame, mustard and poppy and a supplemental nutrient selected from the group consisting of yeast, calcium, magnesium, complex carbohydrates and manganese;
sterilizing said media at a predetermined temperature and for a predetermined time;
cooling said sterilized media to room temperature;
forming a substrate composed of oat hulls, water, maltodextrin and CaSO₄;
inoculating said substrate with a culture of Ganoderma applanatum;
grinding said inoculated substrate into particles;
combining said culture/gelling liquid and said inoculated substrate to form a mixture;
placing said mixture in a mold of predetermined shape;
incubating said mixture to form a member of predetermined shape; and
drying said member to 0% moisture.

10. A method as set forth in claim 9 wherein said culture/gelling liquid is combined with said substrate at a ratio of 1 liter of culture/gelling liquid to 5 liters of substrate.
